(19) United States
(12) Patent Application Publication
(10) Pub. No.: US 2016/0262743 A1
(43) Pub. Date: Sep. 15, 2016

(54) TACK GUIDE WITH LUMENS FOR ARTICULATING MEMBERS

(71) Applicant: Nir Altman, Kibbutz Kfar Etzion (IL)

(72) Inventor: Nir Altman, Kibbutz Kfar Etzion (IL)

(21) Appl. No.: 14/645,575

(22) Filed: Mar. 12, 2015

(51) Int. Cl. A61B 17/068; A61B 17/00; A61B 17/064

(52) U.S. Cl. CPC A61B 17/068; A61B 17/064; A61B 17/00234; A61B 2017/0649; A61B 2017/0647

(57) ABSTRACT

A tack guide is affixed inside a tacker applicator shaft. The tack guide includes a proximal guide portion from which distally extends a distal coil portion. The proximal guide portion includes one or more lumens adapted for passing therethrough articulating members.

### FIELD OF THE INVENTION

The present invention relates generally to tacker devices for applying surgical fasteners, such as rotary tacks, to tissues, such as for hernia repairs and the like, and particularly to a tack guide for use with such a tacker.

### BACKGROUND OF THE INVENTION

A number of surgical, laparoscopic and endoscopic procedures require application of rotary tacks to tissues, such as for hernia repairs and the like.

Tackers for applying such rotary tacks are well known. In a typical tacker, a drive shaft is coupled to a trigger. Operating the trigger causes rotation of the drive shaft. Rotary tacks are rotatingly connected to the drive shaft and held in an articulated applicator arm. Operation of the trigger causes the drive shaft to rotate so as to distally advance the rotary tacks for deployment in tissue. Articulating cables are provided for bending the applicator arm at different angles. The articulating cables are manipulated by a handle of the tacker.

The rotary tacks advance through the end of the applicator arm on account of the inner surface of the arm being internally threaded. The rotary tacks advance by a screwing action—the tacks behave like external threads that threadingly advance through the internal threads. Machining internal threads in the applicator arm, however, is costly and can require special machining techniques. Other techniques, such as welding a coil in a special extruded tube are also very costly and require special jigging for welding.

### SUMMARY OF THE INVENTION

The present invention seeks to provide an improved tack guide for surgical tackers, as is described more in detail hereinbelow. In particular, the present invention seeks to provide a simplified and less expensive construction of the threaded tube of the tack guide. The invention provides a novel (e.g., helical) tack guide with lumens for articulating members, such as articulation cables. The present invention employs an inexpensive tube with additional novel structure that provides an internal thread form and lumens for the articulation cables.

There is thus provided in accordance with a non-limiting embodiment of the present invention a tacker device including a tack guide affixed inside a tacker applicator shaft, the tack guide including a proximal guide portion from which distally extends a distal coil portion, the proximal guide portion including one or more lumens adapted for passing therethrough articulating members.

In accordance with an embodiment of the present invention the distal coil portion includes an internally threaded section formed by helical members spaced axially from one another over a portion of a longitudinal length of the tack guide.

In accordance with an embodiment of the present invention the helical member includes a plurality of helical segments affixed between elongate members. The elongate members may be affixed to an inner wall of the tacker applicator shaft. The elongate members may be affixed to the proximal guide portion. The lumens are bordered by an outer contour of the proximal guide portion and an inner wall of the tacker applicator shaft.

In accordance with an embodiment of the present invention a distal end of the distal coil portion has a keyed shape. The keyed shape can be used to properly register the coil portion's angular orientation with an articulating member at the end of the tacker. Additionally or alternatively, the coil portion's angular orientation can be set with an assembly jig when affixing the coil portion in the tacker applicator shaft tube.

### BRIEF DESCRIPTION OF THE DRAWINGS

The present invention will be understood and appreciated more fully from the following detailed description taken in conjunction with the drawings in which:

FIG. 1 is a simplified, transparent-view illustration of an applicator shaft of a tacker, showing a helical tack guide assembled therein, constructed and operative in accordance with an embodiment of the present invention;

FIG. 2 is a simplified, cutaway illustration of the applicator shaft showing the helical tack guide therein;

FIG. 3 is a simplified pictorial illustration of the helical tack guide; and

FIG. 4 is a simplified end-view illustration of the helical tack guide in the applicator shaft.

### DETAILED DESCRIPTION OF EMBODIMENTS

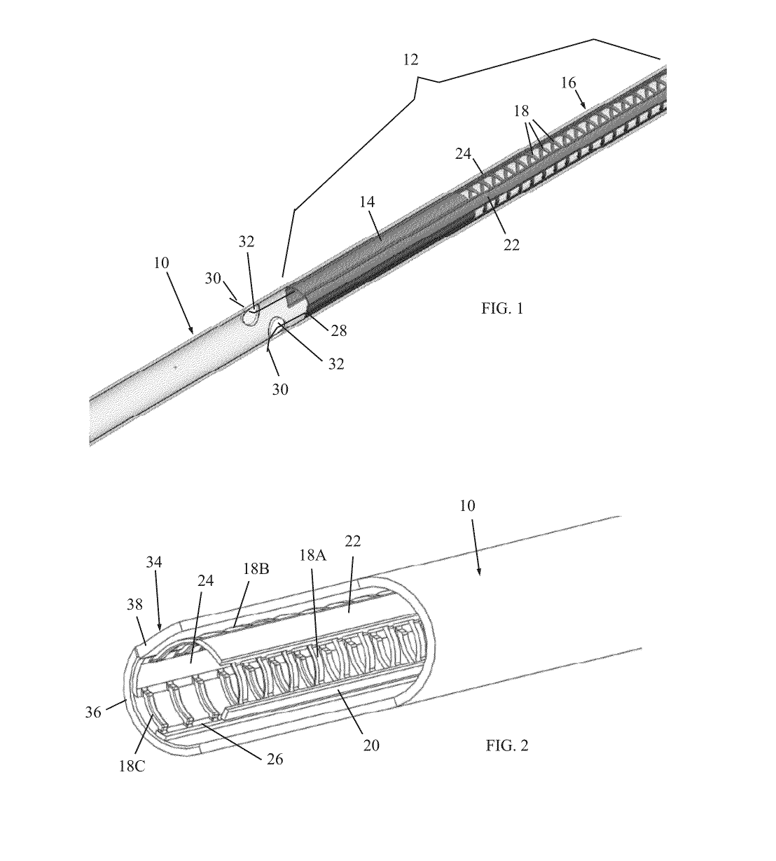

Reference is now made to FIG. 1, which illustrates a tacker applicator shaft 10, constructed and operative in accordance with a non-limiting embodiment of the present invention.

A tack guide 12 (e.g., helical) is affixed inside the tacker applicator shaft (tube) 10. The shaft 10 may be an inexpensive tube made of stainless steel or any other suitable material. The tack guide 12 includes a coil portion 16. The coil portion 16 is an internally threaded section formed by helical members 18 spaced axially from one another over a portion of the longitudinal length of helical tack guide 12. Rotary tacks (not shown) can threadedly advance through this internally threaded section. The helical tack guide 12 may be made of stainless steel or any other suitable material. The tack guide 12 may also include a guide portion 14, proximal to the coil portion 16; however, in some embodiments, the coil portion 16 extends the entire length of tack guide 12 and there is no guide portion 14. Guide portion 14 has no threaded forms and may be used for added strength or for ease of assembly in the tube 10.

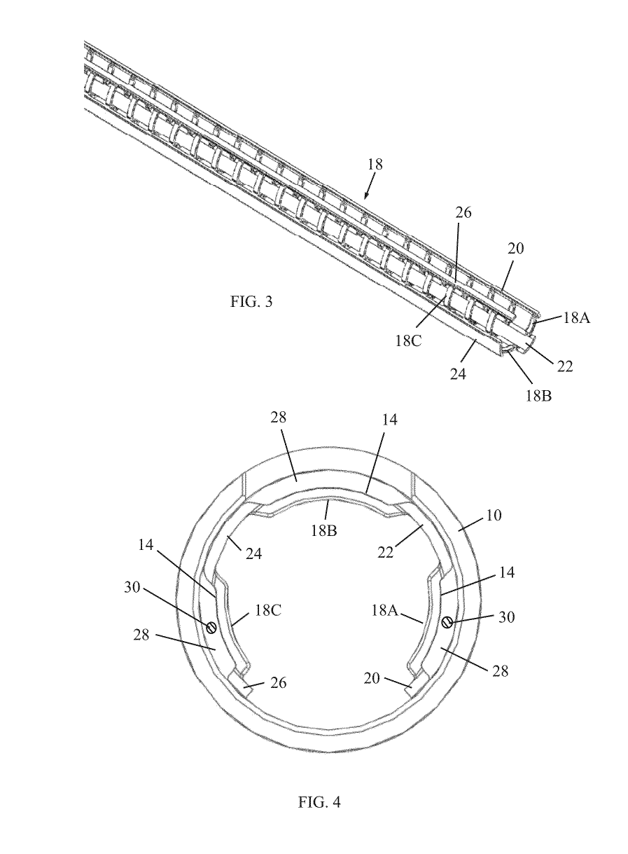

Reference is now made to FIGS. 2 and 3. In the illustrated embodiment, each helical member 18 includes three helical segments 18A, 18B and 18C. Segment 18A is affixed between a first elongate member 20 and a second elongate member 22; segment 18B is affixed between second elongate member 22 and a third elongate member 24; segment 18C is affixed between third elongate member 24 and a fourth elongate member 26. The invention is not limited to this number of segments or elongate members. The assembly of the segments and elongate members may be a one-piece construction or multiple-piece construction. The second and third elongate members 22 and 24 may extend to, and may be adjoined to, the proximal guide portion 14 (as seen in FIG. 1).

As seen in FIG. 4, the tack guide 12 includes one or more lumens or channels 28 through which articulating members 30, such as articulation cables 30, can pass through. Each lumen 28 is bordered by one of the outer contours of the helical segments (18A, B or C), two of the elongate members (20, 22, 24 or 26) and the inner wall of tacker applicator shaft 10. In the illustrated embodiment, there are three lumens 28 (not all of which necessarily have to be used for articulation wires), but the invention is not limited to this number of lumens. If the guide portion 14 is used, then the lumens extend through the guide portion 14.

The elongate members are affixed to the inner wall of tacker applicator shaft 10, such as by welding or bonding or any other suitable joining technique.

The articulation cables 30 can enter apertures 32 (FIG. 1) formed in tacker applicator shaft 10, proximal to the guide portion 14. The articulation cables 30 can extend through the entire length of tacker applicator shaft 10; the proximal ends of articulation cables 30 can be attached to a trigger of the tacker (not shown), and their distal ends can be attached to a bendable applicator member (not shown). The distal end of helical tack guide 12 may abut against this bendable applicator member (not shown). Alternatively, the articulation cables 30 can run the entire length of shaft 10 and not enter any side holes in the shaft 10.

As seen in FIG. 2, the distal end 34 of distal coil portion 16 may have a keyed shape, such as a protruding member 36 with curved sides 38. The keyed shape ensures proper registration with the bendable applicator member, which may have a complementary-shaped keyed end. The keyed shape can be used to properly register the coil portion's angular orientation with an articulating member at the end of the tacker. Additionally or alternatively, the coil portion's angular orientation can be set with an assembly jig when affixing the coil portion in the tacker applicator shaft tube.

It is noted that the invention is not just applicable for a rotary tacker, but may be used for any medical device that requires threaded coils inside a tube. Accordingly, the term “tacker” in the claims is defined as any medical device that requires threaded coils inside a tube.

What is claimed is:

1. A tacker device comprising:
a tack guide affixed inside a tacker applicator shaft, said tack guide comprising a coil portion that comprises an internally threaded section formed by helical members spaced axially from one another over a portion of a longitudinal length of said tack guide, said coil portion being affixed to an inner perimeter of said shaft, and one or more lumens adapted for passing therethrough articulating members, said lumens being located between said helical members and the inner perimeter of said shaft.

2. The tacker device according to claim 1, wherein said tack guide comprises a guide portion proximal to said coil portion.

3. The tacker device according to claim 2, wherein said helical member comprises a plurality of helical segments affixed between elongate members.

4. The tacker device according to claim 3, wherein said elongate members are affixed to an inner wall of said tacker applicator shaft.

5. The tacker device according to claim 3, wherein said elongate members are affixed to said proximal guide portion.

6. The tacker device according to claim 3, wherein said lumens are bordered by an outer contour of said helical members, two of said elongate members and the inner perimeter of said tacker applicator shaft.

7. The tacker device according to claim 1, wherein a distal end of said distal coil portion has a keyed shape.
